(19) United States
(12) Patent Application Publication
(10) Pub. No.: US 2023/0416178 A1
(43) Pub. Date: Dec. 28, 2023

(54) FORMATION OF P-ALKYLPHENOLS BASED ON INTERMOLECULAR REACTION OF ETHYNE WITH 2-ALKYLFURANS IN THE PRESENCE OF GOLD(I) COMPLEXES

(71) Applicant: DSM IP ASSETS B.V., Heerlen (NL)

(72) Inventors: Stephan ACKERMANN-MAURER, Kaiseraugst (CH); Werner BONRATH, Kaiseraugst (CH); Ulla LETINOIS, Kaiseraugst (CH); Thomas NETSCHER, Kaiseraugst (CH); Jan SCHUETZ, Kaiseraugst (CH)

(21) Appl. No.: 18/251,608

(22) PCT Filed: Nov. 8, 2021
(86) PCT No.: PCT/EP2021/080869

§ 371 Date: May 3, 2023

(30) Foreign Application Priority Data
Nov. 9, 2020 (EP) 20206379.8

(51) Int. Cl. C07C 37/14

(52) U.S. Cl. CPC C07C 37/14

(57) ABSTRACT

The present invention relates to the synthesis of p-alkylphenols and of 2,6-dimethyl-4-alkyl phenol from 2-alkylfurans with acetylene in the presence of a gold(I) complex as well as to 2,3,6 trimethylhydroquinone.

### TECHNICAL FIELD

The present invention relates to preparation of p-alkylphenols and 2,3,6-trimethylhydroquinone and as well to the use of Au(I) complexes in its synthesis.

### BACKGROUND OF THE INVENTION

The p-alkylphenols represent a very important class in chemistry. The compounds p-isopropylphenol and p-tert.butylphenol are important intermediates for the synthesis of resins, particularly of phenolic resins but also for polycarbonates. p-Alkylphenols have antiseptic, disinfectant, fungicidal and antioxidant properties. Due to these properties they are broadly used as antiseptics, disinfectants, biocides and antioxidants. Particularly, p-cresol is a very important substance as it used in large scale for the synthesis of 2,6-di-tert.butyl-p-cresol (BHT) which is one of the most important antioxidants in industry. In fragrance industry p-cresol is used to obtain p-cresolcarboxylic acid esters and p-cresol methyl ether being an intermediate of anisaldehyde.

Historically, cresols have been isolated from coal tar. Subsequent synthetic pathways of cresols or alkylphenols in general mainly suffer from the disadvantage that such synthesis yields mixtures of the respective o-, m- and p-isomers. These isomers are difficult to separate. For example, it is known that particularly m- and p-cresol are difficult and costly to be separated.

Syntheses based on any renewable raw materials are very attractive from an ecological point of view as this allows to strongly reduce the dependency of chemical industry from the diminishing fossil oil reserves. Y. Román-Leshkov et al., Nature 2007, 447, 982-985, discloses that 2-methylfuran can be obtained from biomass. Therefore, 2-methylfuran is a very interesting building block to be used in chemical industries.

Furthermore, the use of starting materials from renewable resources are advantageous in providing a better CO₂-balance as compared to starting materials derived from oil.

WO 2016/114668 A1 discloses that furans can be reacted with dienophiles having an electron withdrawing group to yield a phenolic compound having an electron withdrawing group. It discloses in example 1, that p-cresol can be produced by a multistep synthesis from 2-methylfuran and methyl propiolate in the presence of the Lewis acid AlCl₃ followed by hydrolysis and decarboxylation at 170° C. However, this process is rather complex and shows a very low yield (over the 3 steps) of only 19%.

WO 2015/110654 A1 or WO 2015/110655 A1 disclose the use of Au(I) complexes for the synthesis of 2,5-dimethylphenol or 2,3,6-trimethylphenol, resin from 2,5-dimethylfuran and ethyne or propyne, respectively. The methyl groups in 2,5-dimethylphenol or 2,3,6-trimethylphenol are, however, in the ortho or meta position to the phenolic OH group.

2,6-Dimethyl-4-alkyl phenol, and particularly mesitol (2,4,6-trimethylphenol) are important broadly used chemical compounds, particularly to formulate binders such as for printing or paper restauration purposes.

2,3,6-trimethylhydroquinone is a key intermediate in the synthesis of alpha-tocopherol.

A synthetic pathway for p-alkylphenols in general, and for p-cresol, mesitol or 2,3,6-trimethylhydroquinone, respectively, in particular, based on renewable resources is a much desired process in the market.

### SUMMARY OF THE INVENTION

Therefore, the problem to be solved by the present invention is to offer a synthesis of p-alkylphenols which has a very high selectivity and yield.

It has been surprisingly found that the present invention is able to solve this problem. The process of the invention is very unique in that it yields in a very efficient way the targeted p-alkylphenols. It is, furthermore, very advantageous in that the starting materials are sustainable in view that 2-methylfuran can be obtained from renewable biomass and that the gold(I) complex is a catalyst and can be recycled and re-used.

It has been, furthermore, found that by using this approach 2,6-dimethyl-4-alkyl phenols, particularly mesitol, as well as 2,3,6-trimethylhydroquinone can be obtained in a very highly efficient process from 2-methylfuran, which is a biosourced chemical, to offer highly advantageous CO₂-balances in their synthesis.

It is particularly surprising that a process has been found which selectively yields only the targeted p-alkylphenols and not the expected respective o-alkylphenols or the respective m-alkylphenols or a mixture of the respective o-alkylphenols and m-alkylphenols or even a mixture of the respective o-alkylphenols and m-alkylphenols and p-alkylphenols. This process has shown to have such a high selectivity in the p-alkylphenols that o-alkylphenols or m-alkylphenols could not be detected by analytical methods.

Further aspects of the invention are subject of further independent claims. Particularly preferred embodiments are subject of dependent claims.

### DETAILED DESCRIPTION OF THE INVENTION

In a first aspect the present invention relates to a process for manufacturing p-alkylphenol of the formula (I)

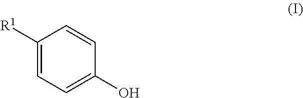

comprising the step of reacting a compound of the formula (II) with ethyne of the formula (III)

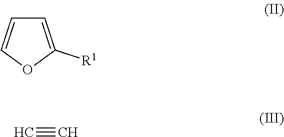

in the presence of at least one Au(I) complex;
and wherein R¹ represents a C₁₋₆-alkyl group.

The term “independently from each other” in this document means, in the context of substituents, moieties, or groups, that identically designated substituents, moieties, or groups can occur simultaneously with a different meaning in the same molecule.

A “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

In case identical labels for symbols or groups are present in several formulae, in the present document, the definition of said group or symbol made in the context of one specific formula applies also to other formulae which comprises said same label.

The expression “process of preparation” is a synonym for “method of preparation” and can be used interchangeable to each other.

The term “inert” in “inert organic solvent”, means that under the conditions of the reaction the solvent undergoes no chemical reaction.

The term “organic ligand” is per se known in complex or coordination chemistry. In the present document an “organic ligand” is an organic molecule that binds to a central metal atom by an electron pair being present on an atom of the ligand. The organic ligand is preferably neutral, i.e. non-charged.

The term “phosphorous containing ligand”, means in this document that the organic ligand comprises at least one phosphorus atom in the chemical structure of the organic ligand.

The process requires a reaction with ethyne of the formula (III). Ethyne, also commonly known as acetylene, is a gas at ambient pressure and temperature.

It is preferred that R¹ is a methyl or ethyl group, particularly a methyl group.

Hence, the most preferred compound of the formula (I) is 4-methylphenol, also known as p-cresol:

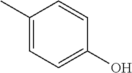

The reaction of ethyne and the compound of the formula (II) takes place in the presence of at least one Au(I) complex.

The gold(I) complex has preferably the formula [Au(I)OL]AN wherein OL represents an organic ligand and AN represents a single charged anion.

The gold(I) complex has preferably a single charged anion (AN) which is selected from the group consisting of [BX₄]⁻, [PX₆]⁻, [SbF₆]⁻, CF₃COO⁻, sulfonates, particularly a sulfonate of the formula (AN-II), tetra(3,5-bis(trifluoromethyl)phenyl)borate (BAr_{F}⁻), tetraphenylborate, and anions of the formula (AN-I)

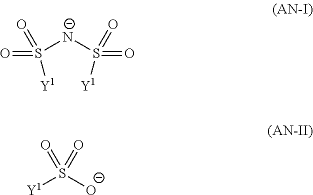

wherein X represents a halogen atom, particularly F or Cl;
and Y¹ represents a phenyl or a C₁₋₈-alkyl group which preferably is substituted by at least one halogen atom.

Preferably Y¹ represents a CF₃ group. So, preferably, the anions of the formula (AN-I) is the anion of the formula (AN-Ia), i.e. the anion of bis(trifluoromethane)sulfonimide, which is also known as triflimidic acid.

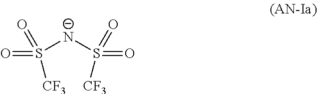 [SMILES: O=S(=O)([N-]S(=O)(=O)C(F)(F)F)C(F)(F)F]

Preferred sulfonates are halogenated anions of organic sulfonic acids, particularly of trifluoromethanesulfonic acid, which is also known as triflic acid. Therefore, the preferred sulfonates are trifluoromethanesulfonates, which are also known as triflates.

In a more preferred embodiment the anion (AN) is an anion which is selected from the group consisting of [SbF₆]⁻, [BX₄]⁻, triflate, and anions of the formula (AN-I). A particularly preferred anion is [SbF₆]⁻.

It is preferred that the gold(I) complex has an organic ligand (OL) which is either
at least one phosphorous containing ligand, particularly a phosphorous containing ligand which is selected from the group consisting of the formula (P1), (P2), (P3), (P4), (P5), (P6), (P7) and (P8);

or
at least an imidazole-2-ylidene ligand, particularly 1,3-bis(2,6-diisopropylphenyl)-1,3-dihydro-2H-imidazol-2-ylidene (=compound of the formula (IM));

or
at least an 1H-1,2,3-triazol ligand, particularly of the formula (TR-1) or (TR-2) or (TR-3), more particularly of the formula (TR-3);

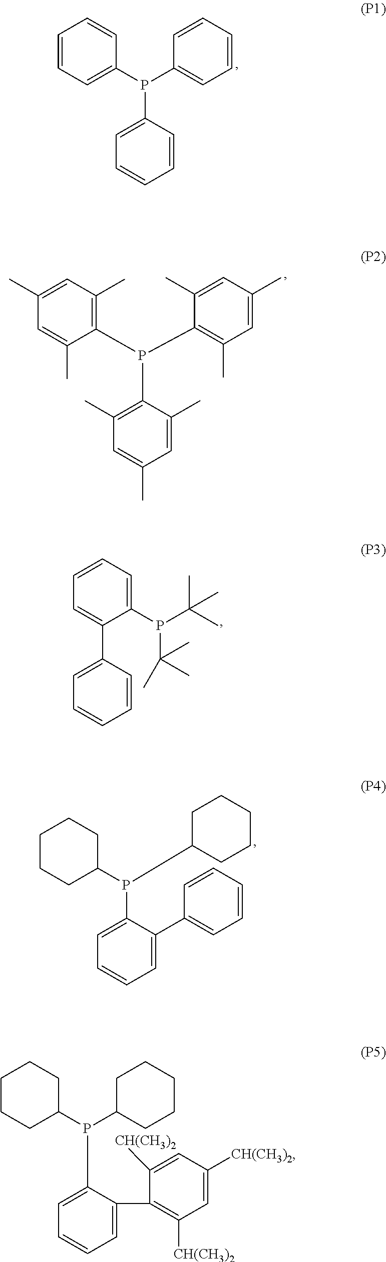

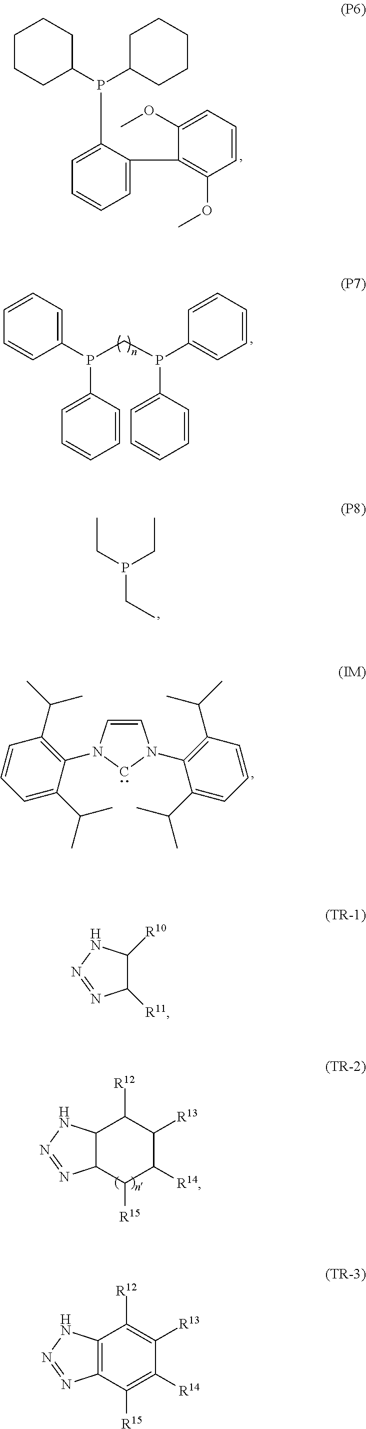

wherein R¹⁰ and R¹¹ represent independently from each other either H or a linear or branched C₁₋₁₀-alkyl or C₄₋₁₀-cycloalkyl group;
and
wherein R¹², R¹³, R¹⁴ and R¹⁵ represent independently from each other H or a linear or branched C₁₋₆-alkyl group;
n stands for an integer of 1-6 and n′ stands for 0 or 1 or 2.

The organic ligand (OL) of the formula (P4) is also known as CyJohnPhos.

The synthesis of these organic ligands (OL) is known to the person skilled in the art.

It is preferred that the Au(I) complex comprises at least a one phosphorous containing ligand.

It is, furthermore, preferred that the Au(I) complex comprises at least 1,3-bis(2,6-diisopropylphenyl)-1,3-dihydro-2H-imidazol-2-ylidene (=compound of the formula (IM)) as ligand.

The Au(I) complex can be added to one or a mixture of the starting material of compound of the formula (II) and/or formula (III) as such, i.e. particularly in the form of a gold(I) complex of the formula [Au(I)OL]AN, or the Au(I)-complex is formed in situ in one of the starting materials or the reaction mixture (before or after the reaction has started).

Particularly, the gold(I) complex is prepared from a gold(I) chloro complex and a silver(I) salt. The silver(I) salt is preferably Ag(I)AN. The organic ligand is in this case either present in the reaction mixture of the gold(I) chloro complex with the silver(I) salt or is part of the gold(I) complex. By this reaction the desired gold(I) complex, i.e. preferably [Au(I)OL]AN, is prepared. AgCl formed by this reaction as precipitate does not interfere negatively with the reaction of preparing the compound of the formula (I).

Hence, the gold (I) complex is preferably of the formula [Au(I)OL]AN wherein OL represents an organic ligand and AN represents a single charged anion and the gold (I) complex is prepared by the reaction of Au(I)OLCl and AgAN.

In a further embodiment the step of reacting a compound of the formula (II) with ethyne of the formula (III) is in the presence of at least one Au(I) complex in combination with at least one Ag(I) salt or Ag(I) complex.

Said at least one Ag(I) salt or Ag(I) complex is preferably AgSbF₆.

Preferred Au(I) complexes of the formula [Au(I)OL]AN are selected from the group consisting of

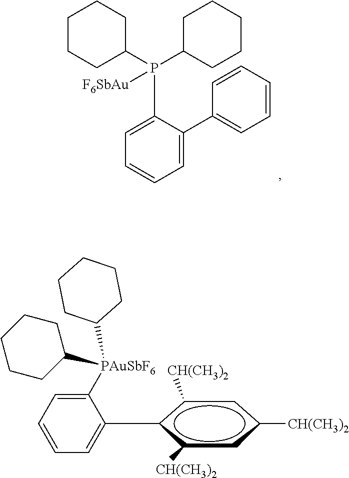

and [Au(I)P6]AN-Ia, wherein P6 is the organic ligand of the formula (P6) and AN-Ia is the anion of the formula (AN-Ia).

In another preferred embodiment, the gold (I) complex is of the formula [Au(I)OL]AN wherein OL represents an organic ligand and AN represents a single charged anion and the gold (I) complex is prepared by the reaction of Au(I)OLCl and NaAN. This reaction is particularly preferred if AN is [BAr^{F}₄]⁻.

In a more preferred embodiment the Au(I) complex is chloro[1,3-bis(2,6-diisopropylphenyl)imidazol-2-ylidene]gold(I).

In the case when the step of reacting a compound of the formula (II) with ethyne of the formula (III) is performed in the presence of at least one Au(I) complex in combination with at least one Ag(I) salt or Ag(I) complex, the molar ratio of Au(I) complex to Ag(I) salt or Ag(I) complex is preferably in the range of 0.4:1 to 3:1, particularly in the range of 0.75:1 to 1.25:1, preferably in the range of 0.9:1 to 1.1:1.

The molar ratio of the compound of the formula (II) to ethyne (of the formula (III)) is preferably in the range of 1:1-1:20, particularly 1:2-1:10.

The gold(I) complex is used typically in a molar ratio of the compound of the formula (II) to the Au(I) complex is in the range of 10′000:1-5:1, particularly of 500:1-5:1, preferably of 100:1-5:1, more preferably of 20:1-10:1.

The compounds of the formula (II) are readily available. Particularly, 2-methylfuran can be obtained from biomass, particularly as disclosed by Y. Román-Leshkov et al., Nature 2007, 447, 982-985. 2-Methylfuran is, therefore, a much appreciated bio-based starting product for any sustainable synthesis of respective targeted end products and is an important factor in realizing low CO₂-balance in the synthesis of chemicals.

The reaction is preferably carried out under normal pressure (i.e. 1013 mbar). The reaction temperature is particularly between 0° and 140° C., more particularly between 10° C. and 80° C., preferably between 15° C. and 35° C. It is very advantageous that the reaction can be performed at low temperatures, particularly at room temperatures.

The reaction of the compound of the formula (II) with ethyne of the formula (III) in the presence of at least one Au(I) complex is preferably made in an inert organic non-nucleophilic solvent or a mixture of inert organic non-nucleophilic solvents. Preferred solvents are halogenated solvents, particularly dichloromethane, 1,2-dichloroethane, chloroform or 2,2,2-trifluoroethanol; or toluene, ethyl acetate or cyclohexanone.

It has been observed that particularly mixtures of dichloromethane and 2,2,2-trifluorethanol, preferably in an excess of dichloromethane, more preferably a mixture of dichloromethane with 5% by volume of 2,2,2-trifluoroethanol, are very suitable for obtaining a high selectivity of the compound of the formula (I).

It is preferred that the organic solvent is a hydrocarbon or a chlorinated hydrocarbon, preferably dichloromethane.

It has been observed that the present reaction surprisingly yields the p-alkylphenol of the formula (I) and not the respective m-alkylphenol or o-alkylphenol, as might be expected by the person skilled in the art from the disclosure of the state of the art documents WO 2015/110654 A1 or WO 2015/110655 A1.

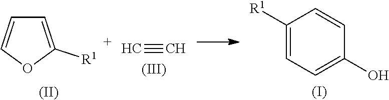

The reaction occurs smoothly in particular high yield and selectivity in the desired product, i.e. the p-alkylphenol of the formula (I). Yield of more than 75%, preferably more than 80%, even more preferred more than 83%, can be achieved. By optimizing the reaction conditions, even higher yields can be obtained.

In a further aspect, the invention relates to a composition which comprises
a) at least one compound of the formula (II)

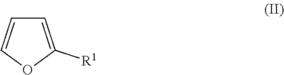

wherein R¹ represents a C₁₋₆-alkyl group.
b) ethyne of the formula (III);

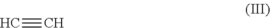

c) at least one Au(I) complex; and
d) optionally at least one Ag(I) salt or Ag(I) complex.

This composition reacts to compounds of the formula (I) as described above in great detail.

As we could show above Au(I) complexes can be used for the synthesis of compounds of the formula (I). Therefore, a further aspect of the resent invention is the use of a Au(I) complex for the synthesis of compounds of the formula (I). Preferred specific embodiments are the ones as already mentioned above.

In a further aspect, the invention relates to a process for manufacturing of 2,6-dimethyl-4-alkyl phenol of the formula (IV) comprising the steps composition which comprises
i) manufacturing p-alkylphenol of the formula (I) as it has been discussed above in great detail

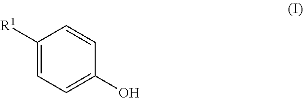

followed by
ii) methylation of the compound of the formula (I) to yield a compound of the formula (IV)

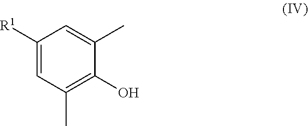

wherein R¹ represents a C₁₋₆-alkyl group.

This process particularly leads to mesitol (2,4,6-trimethyl phenol), i.e. formula (IV) in which R¹ is methyl.

The compound of the formula (I) can be methylated for example in an autoclave with methanol in the presence of lithium hydroxide monohydrate at elevated temperatures as disclosed in EP 1 108 705 A1, particularly by example 3 to yield the compound of formula (IV).

It has been found that the methylation of compound of the formula (I) to yield compound of formula (IV) can be particularly achieved by gas phase methylation, particularly by subjecting the compound of formula (I) to a mixture of methanol and water in the presence of a oxidic catalyst in inert atmosphere at a temperature of between 300 and 500° C.

In an even further aspect, the invention relates to a process for manufacturing 2,3,6-trimethylhydroquinone of the formula (VI) comprising the steps
i) manufacturing a compound the formula (I) as it has been discussed above in great detail

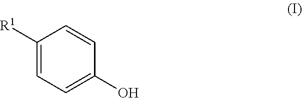

followed by
ii) methylation of the compound of the formula (I) to yield a compound of the formula (IV)

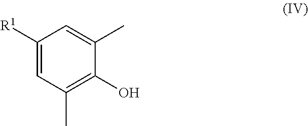

followed by
iii) oxidation of the compound of the formula (IV) to yield a compound of the formula (V)

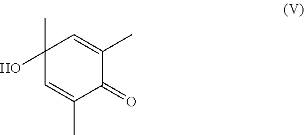

iv) rearrangement of the compound of the formula (V) to yield a compound of the formula (VI)

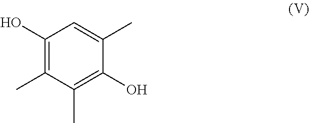

wherein R¹ represents a methyl group.

The transformation of mesitol to 2,3,6 trimethylhydroquinone is principally known to the person skilled in the art, for example from Ullmann's Encyclopedia of Industrial Chemistry, Release 2012, “Vitamins”, vol. 38, pages 204 (DOI: 10.1002/14356007.o27_o07).

The methylation in step ii) has been already discussed above in great detail.

The oxidation of step iii) can be performed by methods principally known to the person skilled in the art.

Particularly, it can be performed by molecular oxygen, particularly in the presence of a cobalt complex and/or in the presence of a base, particular an alkali metal salt, details of which are as disclosed in DE 2 314 600 or DE 2 747 497.

Furthermore, the oxidation of step iii) can be performed by chlorine in a suitable solvent preferably in the absence of a base, followed by hydrolysis with water as described in U.S. Pat. No. 4,612,401.

Furthermore, the oxidation of step iii) can be performed by hypohalo-genous acid or salt in an aqueous medium or a mixture of water and an organic solvent; details of which are as disclosed in EP 0 084 158 A1.

The rearrangement in step iv) can be performed by methods principally known to the person skilled in the art.

Particularly, it can be performed thermally, preferably by heating the compound of the formula (V) to a temperature of about 95° C., preferably in the presence of base, such as sodium hydroxide, such as disclosed in DE 2 314 600.

Furthermore, the rearrangement in step iv) can be performed by heating the compound of the formula (V) to a temperature of at least 100° C. in a non-acid liquid medium selected from the group consisting of methanol and an aqueous medium selected from the group consisting of water and an aqueous solution of a water-soluble organic solvent, details of which are as disclosed in FR 2 200 225 or DE 2 345 062.

2,3,6-Trimethylhydroquinone of the formula (VI) can be smoothly obtained by the present process.

### Examples

The present invention is further illustrated by the following experiments.

### Synthesis of p-cresol

38.0 mg chloro[1,3-bis(2,6-diisopropylphenyl)imidazol-2-ylidene]gold(1) (60 μmol, 0.06 equiv.) and 21.0 mg silver hexafluoroantimonate (60 μmol, 0.06 equiv.) are dissolved in 2000 μL dichloromethane under argon in a 10 ml glass-septum-vial with a magnetic stirring bar. Furthermore, 90.8 μL 2-methylfuran (1 mmol, 1 equiv.) is added. In the acetylene generator acetylene is generated and 130.0 mg of acetylene (5 mmol, 5 equiv.) is bubbled into the reaction over two hours at 23° C. The reaction is followed by GC. After consumption of 2-methylfuran the catalyst is filtered off, the dichloromethane is removed under reduced pressure and the product is analyzed by GC/MS and NMR. The analysis showed a yield of 84% of p-cresol. Neither o-cresol nor m-cresol could been detected by GC/MS. NMR analysis confirmed the structure of p-cresol.

Performing the same reaction in the absence of a gold(I) complex, i.e. with silver hexafluoroantimonate alone or with other metal salts or complexes such as Au(III) (e.g. AuCl₃) or ZnCl₂, CuCl, AuCl, AgBF₄, Cu(OTf)2 (or Cu(OSO₂CF₃)₂) or AgNO₃ did not show any formation of the desired product.

### Synthesis of 2,4,6-trimethylphenol (methylation of p-cresol)

A catalyst (13 g) consisting the oxides of Fe/Si/Cr/K in a molar ratio of 100/2/1/0.1 was placed into a pipe reactor. The reactor was heated to 440° C. (external temperature measurement) under a flow of nitrogen. A mixture of p-cresol, methanol and water (molar ratio: 1:8:1) was pumped through the reactor with 0.1 ml/min. The feed was cooled after the reactor to room temperature and collected in a flask. After 22¾ h the reaction was stopped. During this time 121 g of feed (p-cresol, methanol and water) were pumped through the reactor. Ca. 51 g feed remained in the reactor and the piping. The product mixture was concentrated in vacuo. The remaining residue (23.4 g) contained 89% 2,4,6-trimethylphenol (yield: 89%).

### Characterization of 2,4,6-trimethylphenol

¹H-NMR (300 MHz, CDCl₃): δ (ppm)=2.20 (s, 6H, CH₃), 2.21 (s, 3H, CH₃), 4.45 (s, 1H, OH), 6.77 (s, 2H, CH);

¹³C-NMR (75 MHz, CDCl₃): δ (ppm)=15.9 (CH₃), 20.5 (CH₃), 122.9 (C), 129.2 (CH), 129.4 (C), 149.9 (C).

1. A process for manufacturing p-alkylphenol of the formula (I) 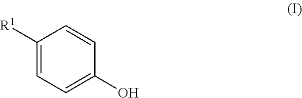
comprising the step of reacting a compound of the formula (II) with ethyne of the formula (III) 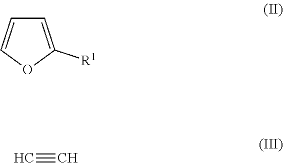
in the presence of at least one Au(I) complex;
and wherein R¹ represents a C₁₋₆-alkyl group.

2. The process according to claim 1, wherein R¹ is a methyl or ethyl group, preferably a methyl group.

3. The process according to claim 1, wherein the Au(I) complex comprises at least a one phosphorous containing ligand.

4. The process according to claim 1, wherein the Au(I) complex comprises at least 1,3-bis(2,6-diisopropylphenyl)-1,3-dihydro-2H-imidazol-2-ylidene as ligand.

5. The process according to claim 1, wherein the Au(I) complex is chloro[1,3-bis(2,6-diisopropylphenyl)imidazol-2-ylidene]gold(I).

6. The process according to claim 1, wherein the step of reacting a compound of the formula (II) with ethyne of the formula (III) is in the presence of at least one Au(I) complex in combination with at least one Ag(I) salt or Ag(I) complex.

7. The process according to claim 5 wherein the Ag(I) salt or Ag(I) complex is AgSbF₆.

8. The process according to claim 6, wherein the molar ratio of Au(I) complex to Ag(I) salt or Ag(I) complex is in the range of 0.4:1 to 3:1, particularly in the range of 0.75:1 to 1.25:1, preferably in the range of 0.9:1 to 1.1:1.

9. The process according to claim 1, wherein the molar ratio of the compound of the formula (II) to ethyne is in the range of 1:1-1:20, particularly 1:2-1:10.

10. The process according to claim 1, wherein the molar ratio of the compound of the formula (II) to the Au(I) complex is in the range of 10′000:1-5:1, particularly of 500:1-5:1, preferably of 100:1-5:1, more preferably of 20:1-10:1.

11. The process according to claim 1, wherein the step of reacting the compound of the formula (II) with ethyne of the formula (III) in the presence of at least one Au(I) complex is made in an inert organic non-nucleophilic solvent or a mixture of inert non-nucleophilic organic solvents, particularly in a hydrocarbon or an chlorinated hydrocarbon, preferably in dichloromethane.

12. The process according to claim 1, wherein the step of reacting the compound of the formula (II) with ethyne of the formula (III) in the presence of at least one Au(I) complex is made at a temperature of between 0° and 140° C., particularly between 10° and 80° C., preferably between 15° C. and 35° C.

13. Composition comprising
a) at least one compound of the formula (II) 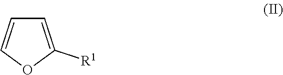
wherein R¹ represents a C₁₋₆-alkyl group.
b) ethyne of the formula (III); 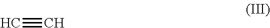
c) at least one Au(I) complex; and
d) optionally at least one Ag(I) salt or Ag(I) complex.

14. Use of a Au(I) complex for the synthesis of compounds of the formula (I) 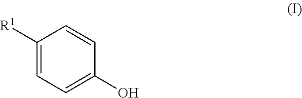
wherein R¹ represents a C₁₋₆-alkyl group.

15. A process for manufacturing of 2,6-dimethyl-4-alkyl phenol of the formula (IV) comprising the steps
i) manufacturing p-alkylphenol of the formula (I) according to a process according to claim 1 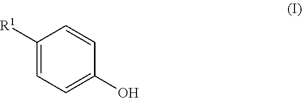
followed by
ii) methylation of the compound of the formula (I) to yield a compound of the formula (IV) 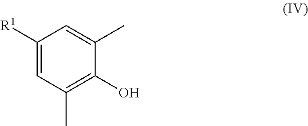
wherein R¹ represents a C₁₋₆-alkyl group.

16. The process of claim 15, wherein the formula (IV) is mesitol (2,4,6-trimethyl phenol).

17. A process for manufacturing 2,3,6 trimethylhydroquinone of the formula (VI)
comprising the steps
i) manufacturing a compound the formula (I) according to a process according to claim 1 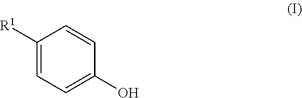
followed by
ii) methylation of the compound of the formula (I) to yield a compound of the formula (IV) 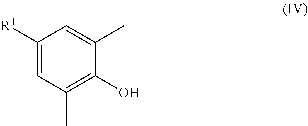
followed by
iii) oxidation of the compound of the formula (IV) to yield a compound of the formula (V) 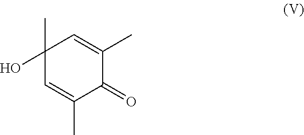
iv) rearrangement of the compound of the formula (V) to yield a compound of the formula (VI) 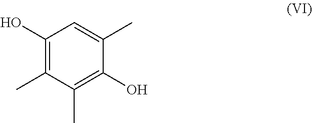
wherein R¹ represents a methyl group.
